(19) United States
(12) Patent Application Publication
(10) Pub. No.: US 2016/0250130 A1
(43) Pub. Date: Sep. 1, 2016

(54) Cosmetic formulation and device for the treatment of deep wrinkles of the skin by means of iontophoresis

(71) Applicant: THE IONTO TEAM, S.L., Barcelona (ES)

(72) Inventors: Javier Marti MAS, Sant Just Desvern (ES); Elisa Suner OLLE, Barcelona (ES)

(21) Appl. No.: 14/974,514

(22) Filed: Dec. 18, 2015

Related U.S. Application Data
(60) Provisional application No. 62/093,623, filed on Dec. 18, 2014.

(51) Int. Cl. A61K 8/73; A61K 8/41; A61K 8/67; A61K 8/34; A61K 8/19; A61K 8/55; A61K 8/49; A61K 8/23; A61K 8/27; A61K 8/36; A61K 8/368; A61K 8/42; A61Q 19/00; A61K 8/60; A61Q 19/08

(52) U.S. Cl. CPC A61K 8/735; A61Q 19/08; A61K 8/41; A61K 8/676; A61K 8/345; A61K 8/34; A61K 8/19; A61K 8/55; A61K 8/498; A61K 8/67; A61K 8/23; A61K 8/27; A61K 8/36; A61K 8/368; A61K 8/42; A61Q 19/007; A61K 8/60; A61K 2800/83

(57) ABSTRACT

The present invention relates to a cosmetic formulation for the treatment of deep wrinkles of the skin and other skin disorders by means of iontophoresis, said formulation comprising the following active ingredients:
dimethylaminoethanol (DMAE) with a tightening and firming effect;
hyaluronic acid with a moisturizing and filling effect; and
vitamin C with an antioxidant characteristic, including its ethyl ascorbic acid form,

The formulation can additionally include one or more other ingredients for the treatment of other disorders, including: magnesium ascorbyl phosphate (MAP), lipoic acid, carnitine, ellagic acid, magnesium ascorbyl phosphate (MAP), vitamin K oxide, Kojic acid dipaimitate, phytic acid, copper sulfate, zinc sulfate, glycolic acid, salicylic acid and potassium azeloyl diglycinate.

### TECHNICAL FIELD

The present invention relates to a cosmetic formulation preferably applicable by means of an iontophoretic patch for the treatment of deep wrinkles of the skin and other skin disorders by means of iontophoresis,

### PRIOR ART

The eye contour area is one of the most delicate parts of the skin, It tends to become dry, thereby leading to the onset of deep wrinkles and expression lines, which are a sign of aging in both men and women. To prevent their onset, there are a series of treatments which slow down the onset of said wrinkles, but none of them assures the elimination thereof in a significant percentage and in a manner that is sustained over time.

One of the easiest ways to treat eye wrinkles is by means of microinjections of botanic toxin or Botox®, The Botox® technique must be applied at the intradermal-surface level in order to be effective. If the wrinkles are particularly deep, can be combined treatment with Botox® and semiablative or non-ablative rejuvenation laser to achieve greater wrinkle removal efficacy. However, the effects of Botox® last for up to four months and the injections can cause a number of side effects which may even be serious.

Another type of treatment for wrinkles appearing in the eye contour area is by means of the use of lotions and cosmetics. These treatments are not as effective, but new treatments and components have been developed and used that have allowed considerably improving their efficacy, Treatments this type can be classified as serums, creams or patches.

Cosmetics used are continuously evolving. One of these examples is the application of iontophoresis technology, whereby the permeability of these active agents on the skin is increased by means of applying a light microcurrent, achieving an effective effect even on the deepest wrinkles.

Different types of natural treatments that allow reducing wrinkles have also been developed, These treatments are usually applied by way of a mask on the contour area, producing effects similar to those produced by cosmetics.

Some of the proposals known in the prior art for the treatment of wrinkles are mentioned below.

Formulations for the Treatment of Expression Wrinkles:

Multiple cosmetics for the treatment of expression wrinkles and anti-age treatment created by a number of companies in a range of formats, such as creams or serums and masks can be found on the market. Among the compositions and active agents studied by certain manufacturers, there are components such as dimethylaminoethanol (DMAE), hyaluronic acid and antioxidants.

Iontophoresis:

Iontophoresis is a painless, non-invasive method using a gentle galvanic electric current to make the ionized water-soluble compounds reach the intact skin and underlying layers in a localized manner. Iontophoresis has become more important in recent years for systemic and topical delivery. It is particularly interesting for the delivery of low molecular weight hydrophilic solutes in a localized action site. It has been observed that the greatest mayor contribution to the flow of ionic molecules is due to iontophoresis, whereas diffusion or electro-osmosis contributes little to the flow of ions.

Iontophoresis is an active way to deliver an active ingredient to the skin and to achieve enhanced cosmetics benefits in wide range of dermal disorders. The use of electric current and of a formulation with active ingredients at a suitable proportion can achieve better results in cases of wrinkles, cellulite and many other dermal conditions.

Vitamin C is well-known for its antioxidant benefits, but it does not readily penetrate the skin. Iontophoresis systems are capable of favoring the absorption thereof and of other active agents with the same situation. Nevertheless, complications in stabilizing these agents have arisen due to their high reactivity.

The system used in iontophoresis consists of a source of electric energy, electrodes and a cream or solution containing the active ingredient suitable for each treatment (see FIG. 3).
Cosmetic patches:

The influence of pharmaceutical technology is evident in the case of cosmetic patches, not as simple cosmetic forms but rather as cosmetic delivery systems. Cosmetic patches today represent a simple, safe and effective form of and cosmetic applications, in theory, cosmetic patches can be used in the same applications as conventional cosmetics.

There are many ways to classify cosmetic patches: by shape (matrix, deposit), applications (moisturizer, anti-wrinkle), materials (synthetic, natural, hybrid), duration of the application (overnight, half-hour).

Patches for the following functions can be found on the market today: skin-lightening, pore-cleaning, blackhead removers, anti-age and anti-wrinkles, lifting effect, anti-cellulite, “fat eaters,” etc.

Micro-Iontophoresis Patches:

Muscular stimulation comprises several levels activating a larger or smaller group of nerve cells; therefore, in macrostimulation many nerve cells are activated, whereas in microstimulation only one or a small group of cells is activated by means of microcannulas or microelectrodes with low-intensity currents.

The microcurrents are characterized they do not act at the level of the organs (muscles or blood vessels), but rather in the cellular and microstructure environment (endothelial cells, myofibrils, etc.), producing two types of effects physiological with potential applications in aesthetics: non-invasive virtual needle, microcirculation or neurostimulation.

A range of possibilities and advantages therefore opens up when investigating in the field of cosmetics, the following standing out:
Drop in percentage of body fat
Increase in tissue tone
Favoring dermocosmetic treatments
Draining and eliminating fluid retentions
Treatment of neurological pain
Increase in muscle tone
Considerably stylizing the figure
Eliminating flaccidity

Ergonomic transdermal patches integrating iontophoresis technology are disposable, single-use, innocuous and environmentally friendly supports which, by means of the electric energy supplied by a battery, allows increasing penetration of the water-soluble active agents, This technology could be compatible with a wide range of cosmetic formulations.

The structure of the mechanism of action is as follows (see FIG. 4):
1. The microcurrent produces microscopic holes in the stratum corneum (the outermost layer of the skin).
2. The current leads the active ingredients to specific areas of the skin.

3. The pad containing biological water (fluid similar to water) acts in contact with the skin, Patent documents U.S. Pat. No. 7,979,117 and U.S. Pat. No. 8,024,033 describe iontophoretic devices. The present inventors have gone one step further and materialized the benefits provided by iontophoresis technology with a system for immediate moisturizing on two levels: on the surface and at a depth, which allows obtaining a deep wrinkle-filling effect.

Description of the Invention

The present invention relates to a cosmetic formulation for the treatment of deep wrinkles of the skin by means of iontophoresis, comprising in a basic formulation the following active ingredients:
dimethylaminoethanol (DMAE) with a tightening and firming effect;
hyaluronic acid with a moisturizing and filling effect; and
vitamin C with an antioxidant characteristic, including its ethyl ascorbic acid form.

For one embodiment, the vitamin C is formed by ascorbyl glucoside, and is at a proportion of between 1% and 3% by weight with respect to the total weight of the cosmetic formulation, preferably at a proportion equal or substantially equal to 3%.

According to one embodiment, the DMAE is at a proportion of between 1% and 5% by weight with respect to the total weight of the cosmetic formulation, preferably at a proportion equal or substantially equal to 5%.

As regards the hyaluronic acid, it comprises, according to one embodiment, a first sodium hyaluronate, at a proportion less than or equal to 1% by weight with respect to the total weight of the cosmetic formulation, and a second sodium hyaluronate, at a proportion between 1% and 2% by weight with respect to the total weight of the cosmetic formulation, where said first and second sodium hyaluronates differ from one another by their molecular weight.

Preferably, the first sodium hyaluronate is at a proportion equal or substantially equal to 0.75% by weight with respect to the total weight of the cosmetic formulation, and the second sodium hyaluronate is at a proportion of 1% by weight with respect to the total weight of the cosmetic formulation.

In addition to the mentioned active ingredients, the cosmetic formulation of the present invention comprises, according to one embodiment, the following components, at the proportions indicated by weight with respect to the total weight of the cosmetic formulation:
deionized water, as a solvent, at a proportion of between 50% and 70%;
glycerine, as a humectant, at a proportion of between 15% and 30%;
triethanolamine, as a pH regulator, at a proportion of between 1% and 5%;
ethoxydiglycol, as a solvent, at a proportion of between 1% and 3%;
a compound formed by water, sodium benzoate and potassium sorbate, as a preservative, at a proportion of between 1% and 2%; and
sodium chloride, as a viscosity controller, at a proportion equal to or less than 1%.

For a preferred implementation of the cosmetic formulation:
the deionized water is at a proportion equal or substantially equal to 50.8%;
the glycerine is at a proportion equal or substantially equal to 30%;
the triethanolamine is at a. proportion equal or substantially equal to 4,35%;
the ethoxydiglycol is at a proportion equal or substantially equal to 3%;
the compound formed by water, sodium benzoate and potassium sorbate is at a proportion equal or substantially equal to 1.2%; and
the sodium chloride is at a proportion equal or substantially equal to 0.9%, p Preferably, the cosmetic formulation of the invention is configured for the treatment of deep wrinkles of the eye contour area.

The invention also contemplates being completed with one or more of the following active ingredients providing additional effects to treat other skin disorders. Examples of skin disorders are included in the following list: aged skin, dry skin, skin damaged by the sun, wrinkles, age spots, different spots produced by hyperpigmentation, melasma, bags, dark circles, acne, cellulite and obesity.

Therefore, for the purpose of additionally providing an antioxidant effect, including one or more of the following ingredients is furthermore proposed:
magnesium ascorbyl phosphate (MAP), lipoic acid, with a lipolytic effect,
carnitine with a lipolytic effect and
ellagic acid, with a skin lightening effect.

A vasodilating effect can he achieved by adding magnesium ascorbyl phosphate (MAP) to the basic formulation, whereas a vasoconstriction effect is provided by means of adding vitamin K oxide,

Said basic formulation can also be complemented by means of one or more of the following active ingredients with a skin lightening effect inhibiting melanin formation:
Kojic acid dipalmitate,
phytic acid,

Astringent characteristics can be conferred to said basic formulation by means of adding copper sulfate or zinc sulfate.

Furthermore, an exfoliating effect can be obtained by means of adding glycolic acid with a skin line reducing effect or by means of salicylic acid with an anti-irritant effect.

The formulation of the invention can further include potassium azeloyl diglycinate with a sebum-normalizing and moisturizing effect.

The typical concentrations for both the topical use and for the proposed iontophoretic treatment of most of the different ingredients mentioned are indicated below:

| | Typical | |
|---|---|---|
| | concentration for | Iontophoretic |
| Ingredient | topical use | concentration |
| Dimethylaminoethanol | 1.0-5.0% | 0.05%-0.25% |
| (DMAE) | | |
| Hyaluronic acid | 0.1%-1.0% | 0.005%-0.05% |
| Vitamin C | 1.0-5.0% | 0.05%-0.25% |
| Glycolic acid | 1.0-5.0% | 0.05%-0.25% |
| Salicylic acid | 0.1-2.0% | 0.005%-0.10% |
| Ethyl ascorbic acid | 2.0-4.0% | 0.1%-0.2% |
| Ellagic acid | 0.5-1.0% | 0.025%-0.050% |
| Ascorbyl glucoside | 1.0-5.0% | 0.05%-0.25% |
| Potassium azeloyl diglycinate | 3.0-10.0% | 0.15%-0.50% |
| Magnesium ascorbyl | 1.0-5.0% | 0.05%-0.25% |
| phosphate (MAP) | | |
| Kojic acid dipalmitate | 1.0-4.0% | 0.05%-0.20% |
| Phytic acid | 0.05-0.5% | 0.0025%-0.025% |
| Copper sulfate | 0.05-0.5% | 0.0025%-0.025% |
| Zinc sulfate | 1.0% | 0.05% |
| Vitamin K oxide | 1.0-5.0% | 0.05%-0.25% |

The formulation of the invention, or any of its variants described above, will be applied by means of a device in the form of a patch like the one described in the mentioned patent document U.S. Pat. No. 7,979,117, including;
a first electrode;
a second electrode;
a power source electrically connected with said first and second electrodes to apply a voltage between them;
a cosmetic formulation according to the invention (in any of its embodiments) arranged or to be arranged on the first electrode; and an electrically conductive hydrogel arranged or to be arranged on the second electrode, where the mentioned device is configured to apply said cosmetic formulation on the skin, by iontophoresis, upon arranging the flexible patch on the skin with the cosmetic formulation and the hydrogel arranged on, respectively, the first and second electrodes.

According to a preferred form of application, the cosmetic formulation is previously arranged on a retaining portion of the first electrode of the mentioned device.

For an alternative embodiment, a container that is independent of the flexible patch structure and contains the cosmetic formulation has been provided, where the first electrode comprises a retaining portion on which a portion of the cosmetic formulation contained in said container can be arranged.

In any case, the mentioned patch can be used to transport any active substance on the skin. Transporting the active substances in the skin is particularly important in the treatment of both cosmetic and dermatological skin disorders. This treatment enables an effective influence on the skin disorder, without excessive systemic exposure to the active substance.

The foregoing and other advantages and features will be better understood from the following description detailed of embodiments in reference to the attached drawings, which must be interpreted in an illustrative and non-limiting manner, in which:

FIG. 1 shows a comparison of the effects of the product called “Ionto-Patch Ojos” after 15 days of use.

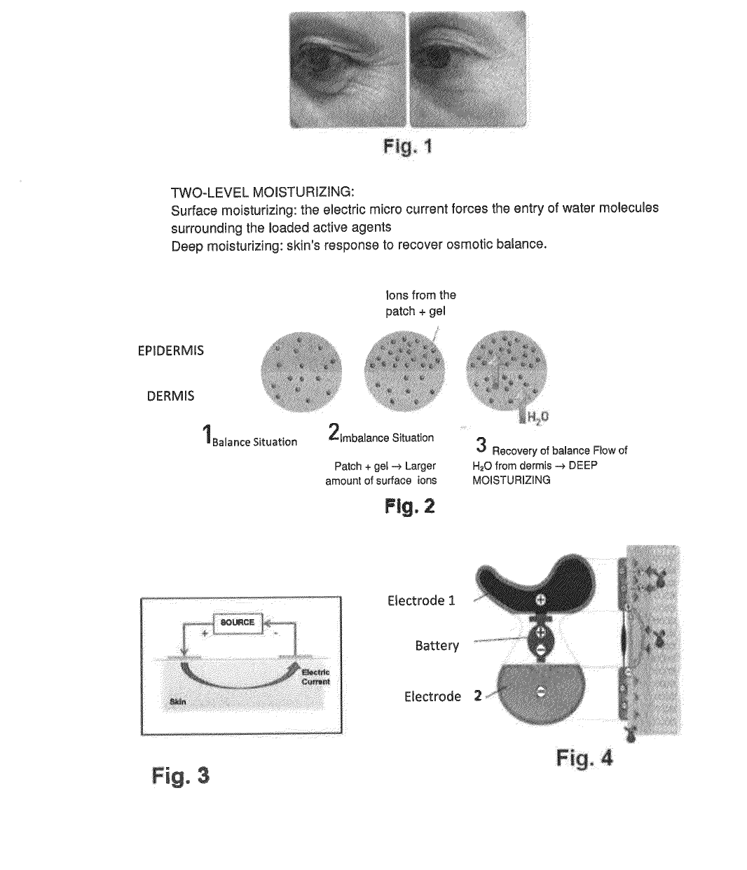

FIG. 2 shows two-level moisturizing achieved as a result of the use of the IONTO-PATCH.

FIG. 3 shows the mechanism of iontophoresis.

FIG. 4 shows a schematic depiction of the device described for applying the cosmetic formulation of the invention for one embodiment, and generally of the mechanism of action of the micro-iontophoresis patches.

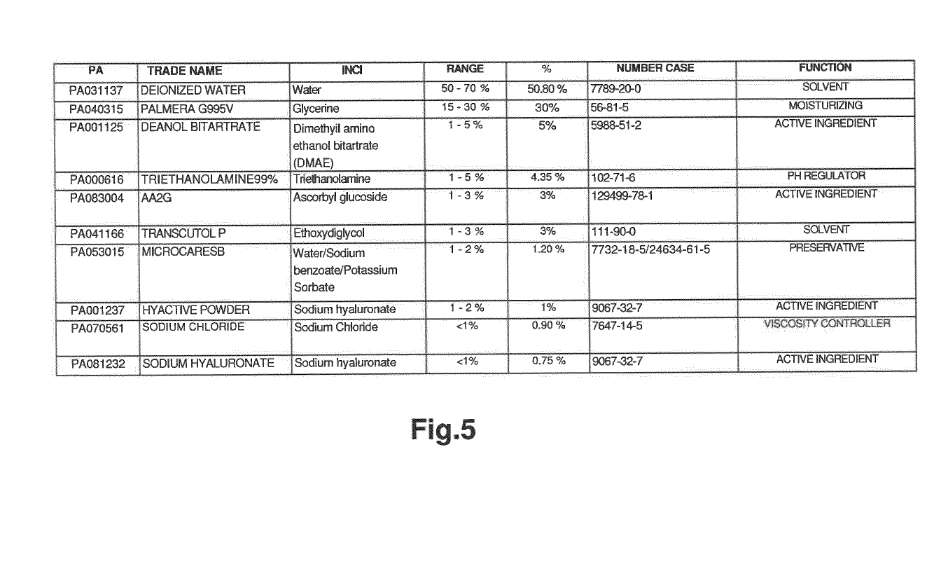

FIG. 5 shows a table describing the composition of the cosmetic formulation proposed by the present invention for the embodiments.

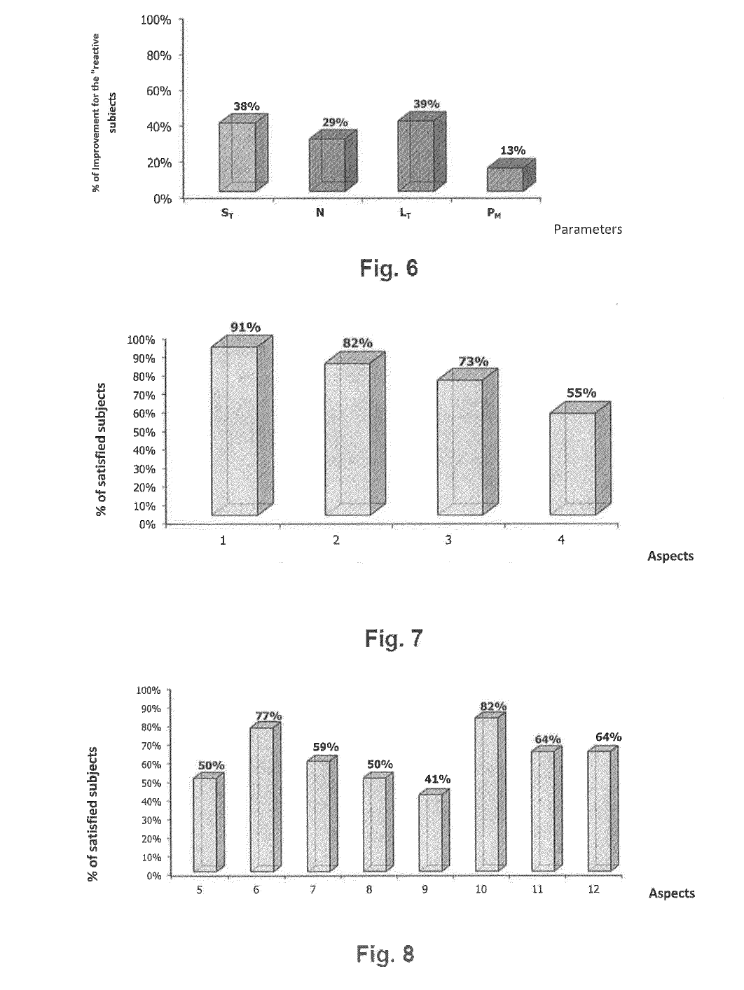

FIG. 6 shows a graphic summary in connection with the assessment of efficacy of the product (device with the cosmetic formulation).

FIGS. 7 and 8 show summarizing graphs of the assessment of efficacy of the composition of the present invention.

### DETAILED DESCRIPTION OF EMBODIMENTS

The mentioned device has been implemented in practice by means of the product sold under the name of “Tonto Patch Ojos,” which is a trans-epidermal patch specific for the treatment of wrinkles in the eye contour area. By means of this product, greater efficacy in a shorter time is sought by means of the increased penetration of the active agents. Non-invasive iontophoresis technology is used to achieve greater penetration of the active agents. The efficacy of the effect, due to the characteristics of the patch and the cosmetic formulation proposed by the present invention, is achieved in about 15 days of treatment (see FIG. 1).

FIG. 4 shows a schematic depiction of the applicator device used in which the first electrode (on which the cosmetic formulation is arranged) has been indicated as “Electrode 1” and the second electrode (on which a electricity-conducting hydrogel is arranged) has been indicate as “Electrode 2.”

By means of this technique, penetration of the active ingredients achieves faster and more effective results. As a result of applying the rnicrocurrent, the opening of pores takes place in a very difficult area due to the fine texture of the skin. Migration of the loaded active agents, which are conducted by the current to the interior, is generated, such that they penetrate at a greater speed and depth.

Furthermore, the occlusive effect of the patch favors the action of the cosmetic formulation, producing three primary effects.
Immediate surface moisturizing because the microelectric current reinforces the entry of water molecules surrounding the loaded active agents.
Deep moisturizing as a response of the skin to recover osmotic balance, The explanation is that before applying the patch there is a situation of balance in the skin; by applying the ions from the patch plus the gel, a situation of imbalance is generated as there is a larger amount of ions on the surface.
The foregoing causes a reaction of the skin to recover the balance, which translates into a flow of water from the dermis, and finally, a deep wrinkle-“filling” effect.

Two-level moisturizing is shown in FIG. 2.

These properties are achieved as a result of combining iontophoresis technology with the specific components of the cosmetic formulation proposed by the present invention which, as indicated hi a preceding section, contains DMAE with a tightening and firming effect, hyaluronic acid with a moisturizing and filling effect, and vitamin C with an antioxidant characteristic. Each of such components is described below.

### DMAE

This is a vitamin B choline analogue and an acetylcholine precursor, and it is becoming increasingly popular as an anti-wrinkle and firming agent. Acetylcholine takes part in the regulation of basic cell functions, such as mitosis, cell contact, cytoskeleton organization, secretion, and absorption, making it interesting on a dermatological level,

It is a basic amine which, considering its alkalinity, is neutralized by acids forming salts that can be used in applications dermatological in the form of topical solutions.

It acts topically on three levels: increasing of acetylcholine release in the dermis (increasing firmness of the skin), increasing water retention in the dermis (temporary tightening effect) and increasing dermal GAGs and collagen synthesis.

The formulation of DMAE, of choline and of acetylcholine is shown below:

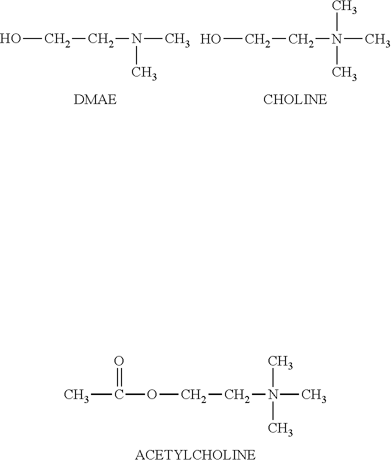

### Hyaluronic Acid

It is a natural polyanionic form, a polysaccharide consisting of N-acetyl-D-glucosamine and β-glucuronic acid. It is present in the intercellular matrix of most connective tissues in vertebrates, particularly in the skin in which it plays a protective role, stabilizing the structure and impact absorption function.

Its viscoelastic nature together with its non-immunogenicity has expanded its use to multiple clinical applications. Its viscoelastic properties act as a stabilizing agent of the cutaneous structure and its hygroscopic properties increase deep and surface skin moisturizing properties.

The organic chemical structure of hyaluronic acid is shown below:

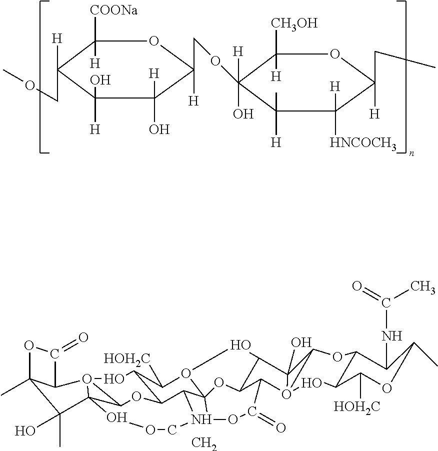

High molecular weight hyaluronic acid does not penetrate the skin as deeply and it has a better moisturizing effect, forming a thin film on the skin (without being occlusive) that prevents dehydration, its ability to retain an enormous amount of water prevents transcutaneous water loss by evaporation and therefore acts as a humectant. It has a high firming and tightening effect and isolating power.

Low molecular weight hyaluronic acid penetrates the skin more deeply and has a greater anti-wrinkle effect. One of its primary functions is to retain water in the intercellular matrix of connective tissue, This water retention ability contributes significantly to elasticity of the skin, it helps to rejuvenate the skin and improve its viscoelastic properties. It is capable of filling in wrinkles from the inside and reducing deep wrinkles and crow's feet.

### Vitamin C (Ascorbic Acid)

Vitamin C is essential in collagen formation due to the fact that it is a necessary component in hydroxyproline and hydroxylysine synthesis in collagen. Hydroxyproline stabilizes the collagen triple helix; the collagen resulting in its absence is structurally unstable and is not segregated from cells at a normal rate, The hydroxylysine is responsible for forming intermolecular cross-linking of collagen.

Ascorbic acid stereoisomers are inactive in the organism, given that the enzymes specifically recognize L-ascorbic acid. L-ascorbic acid has free radical antioxidizing and neutralizing ability: it reduces reactive oxygen production, so the tyrosinase is induced less and less melanin is formed. It is used in cosmetics at a percentage between 5 and 10% with respect to the total composition.

The chemical structure of ascorbic acid is shown below:

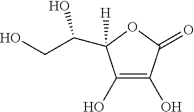 [SMILES: O=C1O[C@H]([C@@H](O)CO)C(O)=C1O]

As there is no formulation on the market to be applied the iontophoresis technology, the present inventors proposed research to obtain an effective formulation to be used by means of a revolutionary cosmetic support for the treatment of wrinkles in the eye contour area and the supralabial area, such that penetration of the water-soluble active agents is increased, achieving production of the cosmetic formulation proposed by the invention.

The cosmetic formulation obtained is shown in the table in FIG. 5, indicating in the column “RANGE” the range of percentages possible for the different components, and in the column “%” the final, exact concentration of the preferred formulation implemented in practice.

The two types of hyaluronic acid indicated in FIG. 5, previously referred to as first and second hyaluronic acids, and the trade names of which are indicated in said table as “HYACTIVE POWDER” and “SODIUM HYALURONATE,” respectively, have the following characteristics.

### “SODIUM HYALURONATE”:

| ASSAYS | LIMITS |
|---|---|
| Loss on drying | <10.0% |
| Purity | >95.0% |
| pH | 5.5-7.5 |
| Total ash | 7.5-12.5% |
| Nitrogen | 3.0-3.8% |
| Total aerobes | <100 cfu/g |
| Fungi and yeasts | <10 cfu/g |
| Mean molecular weight | 1-1.4 MDa |
| Viscosity | >1500 cps |

### “HYACTIVE POWDER”:

| Criteria | Unit | Specification | Method |
|---|---|---|---|
| Origin | — | biotechnological/chemical processing | — |
| Appearance | — | white to creamy fine powder | 206.1.1-012 |
| Appearance of 1% | — | clear to slightly opalescent, | 206.1.1-008 |
| aqueous solution | | colourless solution | |
| Clarity of 1% aqueous | — | ≦0.010 | 206.1.1-008 |
| solution (880 nm, 1 cm) | | | |
| Loss on drying | % | ≦10.0 | 206.1.1-002 |
| Molecular wieght (SEC-MALS) | kDa | 100-150 | 206.1.1-254 |
| pH of 0.5% aqueous solution | — | 5.0-8.0 | 206.1.1-220 |
| Protein* | % | ≦0.20 | 206.1.1-283 |
| Microbial contamination | CFU/g | ≦100 | 206.1.1-800 |
| Uronic acid - UA* | % | ≧45.0 | 206.1.1-005 |
| Sodium hyaluronate - | % | ≧93.0 | 206.1.1-005 |
| UA K 2.067* | | | |
| Ash* | % | ≦10.0 | 206.1.1-014 |
| Preservative | — | none | — |

*calc. on dry basis

The mentioned preferred formulation was subjected to different laboratory tests and tests in a real world setting to demonstrate its efficacy, tests which are described below,
Performing the CHALLENGE TEST:

The challenge test consists of studying the efficacy of antimicrobial conservation based on the European Pharmacopoeia, The test consists of challenging the preparation, if possible in its final container, to determine the resistance of the products to accidental contamination, either during the course of manufacture or in use.

The conservation ability of the formula is estimated by means of the neutralization dilution technique, under given experimental conditions, In this case, the sample (which contains the mentioned preferred formulation) has been inoculated in patches for .iontophoresis referred to as “Ionto-Patch Ojos.” The testing method used follows study protocol PNT-MB-050-08. This protocol indicates that conservation of the formula will be studied by means of the dilution technique. Said study must be carried out by means of experimental conditions determined by the study protocol.

Four different reference strains are used:
A. Pseudomonas aeruginosa ATCC 9027
B. Slaphylocoecus aureus ATCC 6538
C. Candida albicans ATCC 10231
D. Aspergillus brasiliensis ATCC 16404

The media and diluents used in the protocol are as follows:
6.2.1. Fluid for preparation of the bacteria and yeasts suspensions.
6.2.2. Fluid with Tween 80 at 0.05% for preparation of the mold suspension.
6.2.3. Agars for seeding and counting bacteria.
6.2.4. Agars for seeding and counting molds and yeasts.
6.2.5. Fluid for diluting and neutralizing the sample.

Inoculation of the sample with the organism suspension is titrated a 107-108 cfu/ml for each strain separately for the purpose of obtaining a final concentration of 105-106 cfu/ml. In the case of strain D, the standardization will be performed in spores/ml.

The volume of the inoculate suspension will be 1% of the volume of the sample to be inoculated with each strain.

The inoculated sample is kept at 20-25° C., protected from light. Suitable bacteria and fungi culture media (6.2.3 and 6.2.4, respectively) and the neutralizing diluent (6.2.5) for plate counting have been used. The aliquots taken for each control were incubated at 30-35° C. for the case of bacteria and at 20-25° C. for the case of fungi.

Aliquots of the inoculated sample were taken at 0 h, 48 h, 7 days, 14 days and 28 days for bacteria and 0 h, 14 days and 28 days for fungi, after inoculation.

The results obtained in the “challenge test” are divided into two parts which are shown in the following two tables:

TABLE 1
Initial inoculate in the patches called “Ionto-Patch Ojos.”

| Strain | Initial inoculate (cfu/ml) |
|---|---|
| Pseudomonas aeruginosa | 2.4 · 10⁶ |
| Staphylococcus aureus | 5.0 · 10⁶ |
| Candida albicans | 4.9 · 10⁵ |
| Aspergillus brasiliensis | 4.1 · 10⁵ |

TABLE 2
Progression of the amount of microorganisms in the patches
called “Ionto-Patch Ojos.”

| Sample | T | Strain A | Strain B | Strain C | Strain D |
|---|---|---|---|---|---|
| 0786.01-13 | 0 h. | 4.0 · 10⁶ | 4.9 · 10⁶ | 4.0 · 10⁵ | 3.3 · 10⁵ |
| | 48 h. | <10 | 2.4 · 10⁶ | — | — |
| | 7 d. | <10 | 4.6 · 10⁴ | — | — |
| | 14 d. | <10 | <10 | <10 | 2.5 · 10² |
| | 28 d. | <10 | <10 | <10 | <10 |

The analyzed sample (0786.01-13) has an antimicrobial capacity of acceptance B with respect to the Staphylococcus aureus strain and of grade A with respect to the remaining strains.
Evaluation of product efficacy in men:

A study was conducted in order to evaluate the anti-wrinkle effect, to verify cutaneous and ocular acceptability and to assess cosmetic qualities and cosmetic product efficacy, after being used in the provided normal use conditions. Annex C shows this study in greater detail, describing the most relevant results below.

The product anti-wrinkle efficacy was studied in an objective and quantitative manner by means of the technique of taking replicas, before and after treatment, and analyzing the obtained image.

This test consists of the following steps:
Evaluation of anti-wrinkle efficacy.
Cutaneous and ocular acceptability.
Assessment of cosmetic qualities and of the efficacy by way of self-evaluation.

### Evaluation of Efficacy:

The instrumental method chosen to assess efficacy was the replica technique. A number of publications have been studied to determine the cosmetic efficacy, among which the following stand out:
Lévêeque J, EEMCO guidance for the assessment of skin topography, Jour, of the

European Academy of Dermatology and Venereology, 1999. 12, p. 103-114.
Corcuff P., Chatenay F., Brun A., Evaluation of antiwrinkle effects on humans, Int. J. Comet Sci., 1985, 7, p. 117-126.
Corcuff P., De Rigal J., Makki S., et al., Skin relief and aging, J. Soc, Cosmet. Chem., 1983, 34, p. 177-190.

The experimental area was crow's feet, corresponding to the usual area of application of the product.

### Verification of Acceptability:

The chosen method has given rise to a number of publications, among which the following stand out:
Jackson E. M. & Robillard N. F., The controlled use test in a cosmetic product safety substantation program, J. Toxicol. Cut. Ocular. Toxicol., 1982, 20, p.117-132.
Frances Panther M. D., Adverse reactions to eye area cosmetics and their management, J. Soc. Cosmet. Chem., 1982, 33, p. 249-258
Jackwerth B., Krächter H. U., Matthles W., Dermatological test methods for optimising mild tenside preparations, Parfümerie and Kosmetik, 1993, 74, p, 134-141.
Keswick B. H., Ertel K. D., Visscher M. O., Comparison of exaggerated and normal use techniques for assessing the mildness of personal cleansers, J. Soc. Cosm. Chem., 1992, 43, p. 187-193.

The environmental conditions imposed on the subjects for 15 minutes prior to the replica and during same were the following: controlled temperature: T=20° C.±2° C. and controlled relative humidity: RH=45%±15%.

The measurements were taken in a documented and perfectly defined area in a randomly chosen crow's foot. Digital photographs of the experimental areas were also taken by means of the FotoFinder Mediscope system, before and after treatment.

The following parameters were calculated:
N: total number of wrinkles
L_{T}: total length of the wrinkles, expressed in mm
P_{M}: mean depth of the wrinkles, expressed in μm
S_{T}: total surface of the wrinkles, expressed in mm²

The individual results were expressed in absolute values for each experimental time and in percentages of variation in relation to the obtained values. The means and standard deviations were calculated for each parameter and for all the experimental times.
The percentage of “reactive” subjects:
   Subjects in whom (at least) two of the parameters N, L_{T}, S_{T} decreased with a percentage of variation in relation to D0 greater than or equal to 10%: effect on small wrinkles (effect 1).
   Subjects in whom the parameter P_{M} decreases with a percentage of variation in relation to D0 greater than or equal to 10%: effect on deep wrinkles (effect 2). The mean percentage of improvement of cutaneous relief after treatment for the “reactive” subjects and for each parameter related to effect 1 or 2.

For each aspect relating to cosmetic efficacy, the results were subjected to suitable statistical treatment (two-class χ²: comparison of 2 percentages satisfied subjects and unsatisfied subjects).

$${χ}^{2} = \frac{Σ ( {\vert {O}_{i} - {C}_{i} \vert}^{2} )}{{C}_{i}}$$

$${O}_{i} = number observed$$

$${C}_{i} = number calculated$$

$$α = 5 \% risk$$

$$Significant difference if {χ}^{2} > 3.84 ( for α = 5 \% )$$

$$No significant difference if {χ}^{2} \leq 3.84$$

The results of the statistical analysis of the data were done by comparing the values obtained in the treated area before and after treatment by means of the suitable statistical test. For further information, see Annex C.

### Obtained Results

A new product has been obtaining for deep wrinkle-filling treatment, designed to be used twice a week, the primary objective of which is the treatment of wrinkles generated in the eye contour area. The combination of the different active ingredients achieves maximum efficacy as a result of the combination of the designed cosmetic formulation with the device for introducing the formula investigated by the present inventors.

The primary difference in relation to the preceding treatments consists of the use of iontophoresis technology, whereby increasing permeability of the skin, thus being able to act in the deepest wrinkles.

The results obtained in the evaluation test conducted are shown below:

### Evaluation of Anti-Wrinkle Efficacy:

TABLE 3
Summary of treatment efficacy

| Difference D14/D0 in % | | | | | |
|---|---|---|---|---|---|
| Reference | | | | | Type of |
| subject | S_{T} | N | L_{T} | P_{M} | effect |
| 1 | −68 | −29 | −42 | −1 | 1 |
| 2 | 67 | 46 | 29 | −8 | / |
| 3 | 42 | 22 | 31 | 8 | / |
| 4 | −45 | 39 | 46 | −4 | 1 |
| 5 | −23 | −12 | −20 | −10 | 1 + 2 |
| 6 | −23 | −3 | −7 | −2 | / |
| 7 | −13 | −22 | −47 | −7 | 1 |
| 8 | 46 | 4 | 32 | −9 | / |
| 9 | 101 | −9 | 8 | 9 | / |
| 10 | −4 | 23 | −1 | −9 | / |
| 11 | 99 | −7 | 8 | 15 | / |
| 12 | 90 | −7 | 30 | 26 | / |
| 13 | 98 | 43 | 29 | 4 | / |
| 14 | 7 | −14 | −9 | 22 | / |
| 15 | 19 | 13 | 12 | 7 | / |
| 16 | −23 | −21 | −12 | 17 | / |
| 17 | −90 | −19 | −12 | −12 | 1 + 2 |
| 18 | 27 | −17 | −14 | 9 | 1 |
| 19 | −16 | 25 | −4 | −3 | / |
| 20 | 26 | 28 | 26 | 11 | / |
| 21 | −5 | −27 | −4 | −1 | / |
| 22 | 39 | 22 | −2 | −17 | 2 |

| | |
|---|---|
| % of “reactive” subjects (effect 1 and/or 2) | 36% |
| % of “reactive” subjects: effect 1 | 12% |
| % of “reactive” subjects: effect 2 | 14% |

FIG. 6 shows a graphic summary of the assessment of the efficacy of the present invention, where:
N: total number of wrinkles
L_{T}: total length of the wrinkles expressed in millimeters
P_{M}: mean depth of the wrinkles expressed in micrometers
S_{T}: total surface of the wrinkles expressed in square millimeters

### Cutaneous and Ocular Acceptability:

The following tables show the results of the verification of this acceptability.

TABLE 4
Verification of cutaneous acceptability

| | % of | | |
|---|---|---|---|
| | subjects | | |
| Type of | showing | Type of | % of subjects |
| cutaneous | clinical signs | feelings of | showing feelings |
| reactions | attributable | discomfort | of discomfort |
| attributable to the | to the | attributable to the | attributable to the |
| tested product | tested product | tested product | tested product |
| None | 0% | None | 0% |

TABLE 5
Verification of ocular acceptability

| Type of | | | |
|---|---|---|---|
| observed | % of subjects | | % of subjects |
| ocular reactions | showing clinical | Type of feelings | showing feelings |
| attributable to | signs attributable | of discomfort | of discomfort |
| the tested | to the | attributable to the | attributable to the |
| product | tested product | tested product | tested product |
| None | 0% | Slight burning of | 5% |
| | | the eyelids | |
| | | (ref. 13) | |

### Assessment of Cosmetic Qualities:

FIGS. 7 and 8 show a summary of the assessment of efficacy of the composition of the present invention, applied by means of the mentioned device, where:
1 General application of the product on the patch
2 Application of the patch on the skin
3 Application of the patch is comfortable
4 No residues left after applying the patch
5 Firms skin
6 Leaves skin softer and smoother
7 Leaves skin more flexible and elastic
8 Leaves skin with a younger look
9 Reduces wrinkles in the eye contour area
10 Does not leave an oily feeling after application
11 Does not leave a sticky feeling after application
12 Leaves a comfortable feeling

Furthermore, a significant number of subjects consider that it leaves the skin softer and smoother and does not leave an oily feeling after application,

Under the experimental conditions used and taking into account the evaluation scale established by the research center, as well as the evaluated instrumental parameters, it is concluded that the product of the present invention (i.e,, the cosmetic formulation proposed by the invention applied by means of the mentioned device) shows very good acceptability, Its excellent cutaneous tolerance, good ocular acceptability and 36% anti-wrinkle effect are therefore confirmed.

Therefore, as general results of the product, optimal results are achieved with a 15-day treatment, obtaining the following results:
Opening of the pores enabled by applying microcurrent on the skin.
A migration of loaded active agents, which are conducted by the current to the interior of the skin, such that it is penetrated at a very high speed and considerable depth.
Occlusive effect of the patch favoring absorption of the active ingredients formulated in the gel.

The developed treatment is effective in both men and women.

1. A cosmetic formulation for the treatment of deep wrinkles of the skin and other skin disorders by means of iontophoresis, comprising the following active ingredients:
dimethylaminoethanol (DMAE) with a tightening and firming effect;
hyaluronic acid with a moisturizing and filling effect; and
vitamin C with an antioxidant characteristic, including its ethyl ascorbic acid form.

2. The formulation according to claim 1, further comprising one or more of the following active ingredients with an antioxidant effect:
magnesium ascorbyl phosphate (MAP),
lipoic acid with a lipolytic effect,
carnitine with a lipolytic effect, and
ellagic acid with a skin lightening effect.

3. The formulation according to claim 1, further comprising one of the following active ingredients:
magnesium ascorbyl phosphate (MAP) with a vasodilating effect, and
vitamin K oxide with a vasoconstriction effect.

4. The formulation according to claim 1, further comprising one or more of the following active ingredients with a skin lightening effect inhibiting melanin formation:
kojic acid dipalmitate,
phytic acid.

5. The formulation according to claim 1, further comprising one or more of the following active ingredients with astringent characteristics
copper sulfate, and
zinc sulfate.

6. The formulation according to claim 1, further comprising one or more of the following active ingredients with exfoliating characteristics:
glycolic acid with a skin line reducing effect, and
salicylic acid with an anti-irritant effect.

7. The formulation according to claim 1, further comprising potassium azeloyl diglycinate with a sebum-normalizing and moisturizing effect.

8. The formulation according to claim 1, where the vitamin C is formed by ascorbyl glucoside and is at a proportion of between 1% and 3% by weight with respect to the total weight of the cosmetic formulation.

9. The formulation according to claim 8, where the ascorbyl glucoside is at a proportion equal or substantially equal to 3% of the total cosmetic formulation.

10. The formulation according to claim 1, where the DMAE is at a proportion of between 1% and 5% by weight with respect to the total weight of the cosmetic formulation.

11. The formulation according to claim 10, where the DMAE is at a proportion equal or substantially equal to 5% by weight with respect to the total weight of the cosmetic formulation.

12. The formulation according to claim 1, where the hyaluronic acid comprises a first sodium hyaluronate, at a proportion less than or equal to 1% by weight with respect to the total weight of the cosmetic formulation, and a second sodium hyaluronate at a proportion between 1% and 2% by weight with respect to the total weight of the cosmetic formulation, where said first and second sodium hyaluronates differ from one another by their molecular weight.

13. The formulation according to claim 12, where the first sodium hyaluronate is at a proportion equal or substantially equal to 0.75% by weight with respect to the total weight of the cosmetic formulation, and the second sodium hyaluronate, in powder form, at a proportion of 1% by weight with respect to the total weight of the cosmetic formulation.

14. The cosmetic formulation according to claim 1, comprising, in addition to the mentioned active ingredients, the following components, at the proportions indicated by weight with respect to the total weight of the cosmetic formulation:
deionized water, as a solvent, at a proportion of between 50% and 70%;
glycerine, as a humectant, at a proportion of between 15% and 30%;
triethanolamine, as a pH regulator, at a proportion of between 1% and 5%;
ethoxydiglycol, as a solvent, at a proportion of between 1% and 3%;
a compound formed by water, sodium benzoate and potassium sorbate, as a preservative, at a proportion of between 1% and 2%; and
sodium chloride, as a viscosity controller, at a proportion equal to or less than 1%,

15. The cosmetic formulation according to claim 14, where:
the deionized water is at a proportion equal or substantially equal to 50.8%;
the glycerine is at a proportion equal or substantially equal to 30%;
the triethanolamine is at a proportion equal or substantially equal to 4.35%;
the ethoxydiglycol is at a proportion equal or substantially equal to 3%;
the compound formed by water, sodium benzoate and potassium sorbate is at a proportion equal or substantially equal to 1.2%; and
the sodium chloride is at a proportion equal or substantially equal to 0.9%
